(19) United States
(12) Patent Application Publication
(10) Pub. No.: US 2025/0244235 A1
(43) Pub. Date: Jul. 31, 2025

(54) PROCESS OF ACQUIRING A SPECTRUM OF A CROP SAMPLE IN THE FIELD

(71) Applicant: PICKETA SYSTEMS INC., Fredericton (CA)

(72) Inventors: Dominic LEVESQUE, Saint-André (CA); William ROSSIGNOL, Edmundston (CA)

(21) Appl. No.: 18/618,563

(22) Filed: Mar. 27, 2024

(30) Foreign Application Priority Data
Jan. 25, 2024 (CA) 3227313

(51) Int. Cl. G01N 21/3563; G01N 21/359; G01N 33/00

(52) U.S. Cl. CPC G01N 21/3563; G01N 21/359; G01N 33/0098; G01N 2201/127

(57) ABSTRACT

There is disclosed a method for acquiring a spectrum of a crop sample using an acquisition system having a computer, a spectrometer and a user interface including a display device. The method includes, via the display device, prompting a user to input a crop information, the crop information including a type of crop, receiving crop information from the user interface, via the user interface, prompting the user to scan the crop sample with the spectrometer and acquiring a spectrum of the crop sample via the spectrometer.

### FIELD

The improvements generally relate to crop sample analysis, and more specifically to a process of acquiring spectra of crop samples in the field in an interactive manner via a user interface of an image acquisition system.

### BACKGROUND

Agriculture makes a significant use of fertilizers for reasons such as optimizing yield. Fertilizers consist of nutrients that the crops need to develop to their full potential. There are various types of nutrients and different types of crops that may need different amounts of different types of nutrients. While the nutrients are typically already present in the ground to a certain extent, the amount present naturally in the ground is typically insufficient to allow the crops to reach their full potential or optimized yield. However, while fertilizers have significant benefits in agriculture, they are also a significant source of cost. Providing an excessive amount of fertilizer to soil where crops grow can thus represent a loss of profit for the farmers, in addition to potentially having negative effects on the environment.

There is thus a motivation for farmers to provide not only enough fertilizers for their crops, but also just enough fertilizers for their crops. While this objective may appear simple at first glance, there are various challenges to achieving it in practice, which can lead to excessive or insufficient use of fertilizers. Indeed, to provide “just enough” fertilizers for their crops, farmers need to know how much fertilizer their crops need. Farmers, based on their experience and just by looking at them, can sometimes tell that a given crop would benefit from a certain amount of a certain type of fertilizer. However, in practice, this method is often inaccurate. An alternative is to take samples of the crop in the field, and to bring these samples to a laboratory which may take measurements of the levels/concentration of nutrients in the samples. While this may lead to a greater accuracy than a farmer's experience-based assessment, there may be a deterioration of the sample between the harvesting of the sample and the moment when the measurement is made, which may bias the results and introduce a source of inaccuracy. Moreover, this process is relatively tedious and time-consuming. For instance, nutrient concentrations can vary depending on the location on the field, and to be relevant, several samples may need to be taken from different locations on the field. There can be confusion, once the results of the laboratory analysis are received, as to which results correspond to which location on the field. Indeed, different samples may become mixed up during collection or during transport, or even during testing, and the process of correctly grouping and identifying samples can be tedious and cumbersome, let alone the inconveniences of the transport and of the delay between the collection of the samples and the receipt of the analysis results. The delay between sending samples to the laboratory and receiving results thus prevents timely fertilizer applications, as in this case the needs of the crop at the moment where the results are received do not necessarily match the needs of the crop at the moment where the samples were taken.

Accordingly, while known techniques of obtaining indications of the nutrient levels of the crop at different locations were satisfactory to a certain degree, there remains significant room for improvement, and such improvements would likely be correlated to a better use of fertilizers, such as a reduction in the use of fertilizers and associated cost savings and environmental benefits, or an increase in the use of fertilizers which would have a direct effect on yield and thus be a source of profit.

### SUMMARY

One potential technique for performing nutrient level measurement is via spectral analysis. Spectral analysis can involve the acquisition of a spectrum of the crop which can be performed via a spectrometer. Spectrometers are specialized pieces of equipment and typically need to be used by trained technicians, as in particular, image quality may depend strongly on how closely a calibration protocol was followed and/or how the spectrometers are used. Moreover, as there may be fluctuations in the spectra of a given crop from a given location on the field even when performed by a trained technician, it is typically helpful to take a certain number of spectra of crops from a given location of the field in a manner to allow to obtain a suitably reliable indication of nutrient levels at that location. Various techniques of spectral analysis may be used, and the use of machine learning algorithms, often referred to as “artificial intelligence” or AI, may yield good results. Although such techniques may be performed by trained technicians in laboratories, it will be understood that there may be a significant incentive for the process to be performed directly by the farmer in the field.

It was found that at least in some embodiments, and when appropriately guided by hardware and a user interface, it was possible for average farmers to acquire spectra of good quality and reliability, thereby avoiding the need for manually labelling, handling, and transporting the samples between the field and the laboratory. Moreover, by using a machine learning software to perform the nutrient level measurements, the recourse to the laboratory may be avoided altogether. In particular, by capturing all relevant metadata at the time of acquiring the spectra in the field, and performing the machine learning software in the “cloud”, travelling can be avoided altogether. To this end, the farmer may be guided during spectrum acquisition, potentially involving calibration, which can help yield spectra of a suitable level of quality and reliability on average to make the process relevant for large scale application. As such, the farmers using the present technology are assisted in taking measurements that are often considered complex, thereby reducing the risks of taking bad measurements and removing the need of relying on staff trained in using field laboratories.

In accordance with one broad aspect, there is provided a method for acquiring a spectrum of a crop sample using an acquisition system having a computer, a spectrometer and a user interface including a display device, the method comprising, via the display device, prompting a user to input a crop information, the crop information including a type of crop, receiving crop information from the user interface, via the user interface, prompting the user to scan the crop sample with the spectrometer and acquiring a spectrum of the crop sample via the spectrometer.

In one embodiment, the display device further includes a graphical user interface, and at least one of said prompting the user to input a crop information and said prompting the user to scan the crop sample is performed via the graphical user interface.

In one embodiment, said prompting a user to input a crop information further comprises prompting the user to select a location amongst a plurality of predetermined locations.

In one embodiment, said prompting the user to input the crop information further comprises prompting the user to select the type of crop amongst a plurality of predetermined types of crops.

In one embodiment, the method further comprises prompting the user to calibrate the spectrometer prior to said prompting the user to scan the crop sample, the calibration comprising obtaining at least one of an integration time, a dark baseline and a white baseline for the spectrometer.

In one embodiment, the method further comprises combining the spectrum with the crop information to obtain a contextual spectrum.

In one embodiment, the spectrometer is configured to operate in a spectral band defined between about 350 nm and 2,500 nm.

In one embodiment, the prompting the user to scan the crop sample comprises prompting the user to scan the crop sample more than once, thereby obtaining a plurality of spectra of the crop sample.

In one embodiment, the method further comprises activating a lamp prior to said prompting the user to scan the crop sample, and prompting the user to wait for a predetermined amount of time until parameters of the lamp stabilize.

In one embodiment, the method further comprises prompting the user to recalibrate the spectrometer once a recalibration condition has been obtained.

In one embodiment, the recalibration condition comprises at least one of a predetermined amount of time that passed after a previous calibration, a predetermined number of spectra acquired after the previous calibration and a detection of an outlier condition.

In one embodiment, said combining the spectrum with the crop information is performed by embedding metadata corresponding to the crop information in a file that includes the spectrum.

In one embodiment, said acquiring the spectrum comprises combining the plurality of spectra of the crop sample.

In one embodiment, said acquiring the spectrum comprises selecting one or several desired spectra amongst the plurality of spectra of the crop sample, the desired spectrum/spectra having spectral properties that correspond at least partially to a desired spectrum.

In one embodiment, the method further comprises providing the contextual spectrum to a server, the server being configured for analyzing the contextual spectrum and obtaining conditions of the crop therefrom.

In one embodiment, the conditions of the crop comprises at least one of a type and concentration of at least one type of nutrient, a presence of disease and a stage of growth.

In one embodiment, the method further comprises receiving predetermined crop information from the server, wherein said prompting a user to input a crop information is based on the predetermined crop information.

In one embodiment, the method further comprises storing the spectrum on a storage device prior to said providing the spectrum to the server.

In one embodiment, the crop information comprises research data for the crop sample.

In one embodiment, the spectrometer includes two or more spectrometers each covering at least partially a respective spectral band.

Many further features and combinations thereof concerning the present improvements will appear to those skilled in the art following a reading of the instant disclosure.

### DESCRIPTION OF THE FIGURES

In the figures,

FIG. 1 is a front view of a field user device, in accordance with an embodiment;

FIG. 2 is a schematic diagram of a system for acquiring a spectrum of a crop sample using the field user device of FIG. 1, in accordance with an embodiment;

FIGS. 3A-3J show various exemplary frame windows of a field user interface, in accordance with one embodiment;

FIG. 4 is a flowchart of a method for acquiring a spectrum of a crop sample, in accordance with one embodiment; and

FIG. 5 is a block diagram illustrating an example computing device, in accordance with one embodiment.

### DETAILED DESCRIPTION

Described below are example methods and example systems for acquiring spectra of crop samples. The spectra may be analyzed to acquire measurements or indications associated to conditions of the crop, and the results from the analysis can be provided to users. It will be appreciated that the process can be performed via various processing devices, and that the focus of the examples presented below is made on the frontend of the system. In the following description, the term “frontend” corresponds to part of the system with which the user interacts directly. Conversely, the term “backend” corresponds to the part of the system that is not directly accessed by the user, typically tasked for storing and manipulating data.

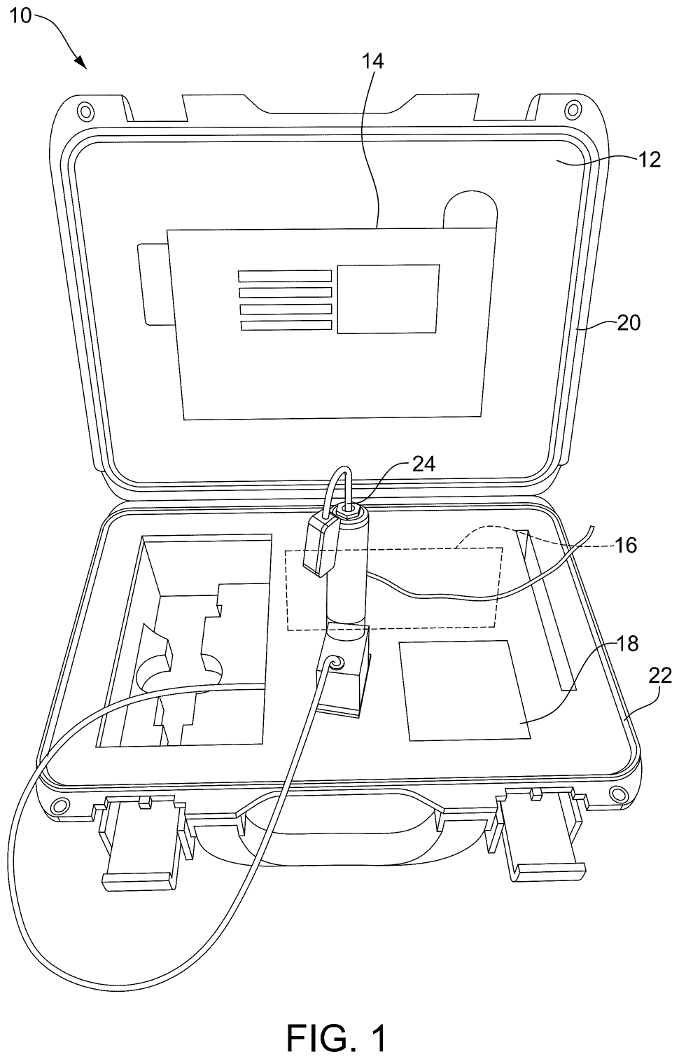

Referring now to FIG. 1, there is shown a front view of a field user device 10. It will be appreciated that the field user device 10 is a device suitable for implementing the methods proposed herein. The field user device 10 has a casing 12 containing a computing device 14, a spectrometer 16 and a sample receiving area 18. The computing device 14 has a user interface. The user interface may include one or more elements depending on the embodiment, and may include, for instance, a display screen, a touch screen, an audible signal emitter, a visual signal emitter, a keyboard, a mouse, etc. In the example presented, the user interface includes a display device that may be configured to display a graphical user interface (GUI). The computing device 14 further comprises a processing unit and a non-transitory computer-readable memory having stored thereon program instructions executable by the processing unit for acquiring spectra of crop samples. The casing 12 may be composed of a top portion 20 that houses the computing device 14 and a bottom portion 22 that houses the spectrometer 16 and defines the sample receiving area 18. In some cases, the top portion 20 and the bottom portion 22 are attached via a hinge so that the casing 12 can be opened for use. In the opened configuration, the top portion 20 is upright and faces the user, such that the display of the computing device 14 is placed in front of the user. The bottom portion 22 can be placed on the ground and planar therewith, such that the sample receiving area 18 is parallel with the ground. It will be appreciated that while the field user device 10 is generally suited to be used in the field where crops are grown, other areas should be contemplated in which the field user device 10 is used for similar purposes, such as in a greenhouse, a laboratory, an indoor field and any other suited area. The term “sample” referred to herein may correspond to one or more portions of the studied crop, such as leaves, stems and other portions of the like.

The display device of the computing device 14 can be embedded within the top portion 20 of the casing, and can include a touchscreen for the user to be able to interact with the computing device 14. The computing device 14 may alternately be equipped with a mouse or other pointer-type input devices for interacting with the user. In operation, the computing device 14, as further described below, is configured to prompt the user for scanning a crop sample by placing the sample on the sample receiving area 18 and scanning the sample with a probe 24 coupled with the spectrometer 16. The output from the spectrometer in that case is generally referred to herein as a spectrum of the sample. In some embodiments, a probe 24 suited for the field user device 10 is an instrument configured to position the end of an optic fiber at a preferred angle and distance with respect to the sample when performing scans. The probe 24 can also be equipped with a lamp for illuminating the sample with light having spectral bands that correspond to the spectral bands of the spectrometer. The probe 24 may define an opening for the light generated by the lamp to be provided to the sample, and for the light obtained from the sample to be provided to the spectrometer 16. In some embodiments the light can be guided with optic fibers between the probe 24, the lamp and the spectrometer 16.

A spectrometer 16 suited for the application is able to cover the spectral band defined between about 350 nm and 2,500 nm, and may thus include measurements of wavelengths in the near-infrared portion of the electromagnetic spectrum in addition to wavelengths in the visible portion of the electromagnetic spectrum. It will be appreciated that some spectrometers may not cover such a large band. As such, two, or more spectrometers 16 having non-overlapping or partially non-overlapping bandwidths may be used complementarily to cover the desired spectral bands. In some cases, the spectral band of interest is defined between about 350 nm and 1,700 nm. In some other cases, the spectral band of interest is defined in the mid-infrared (MIR). The term spectrum generally refers to a spectral measurement of the crop sample. It will be appreciated that the spectroscopy technique used to obtain the spectra may vary, and may include, but is not limited to, near-infrared (NIR) spectroscopy, MIR spectroscopy, Raman spectroscopy, UV-visible spectroscopy, and/or the combination thereof.

In some embodiments, the spectrum may be a spectral image, which is a bidimensional image of a crop sample in which wavelengths outside the visible spectrum may also be captured. The spectrum may include spatial distribution information, e.g. more than one pixel, and in practice, the spectrum may include a large number of pixels. In an alternate embodiment, the spectrum may not include spatial distribution information and only a blended amplitude distribution covering various wavelengths within the bandwidth. In some embodiments, the probe 24 may be configured to scan a 2D surface of the crop sample, using rasterization or other suited techniques. It will be appreciated that the term “scan” referred to herein is representative of causing the spectrometer to measure a spectrum of the sample.

The sample receiving area 18 is generally defined by a surface with low reflectivity across the spectral band of interest. As such, the sample receiving area 18 usually generates low signal when scanning a sample with the probe 24. The sample receiving area 18 is generally sized and shaped for receiving a leaf or a crop sample of similar dimensions. In operation, the user places the sample onto the sample receiving area 18 and scans the sample using the probe 24. While the sample receiving area 18 is defined on the bottom portion 22 of the casing, in some embodiments, the sample receiving area 18 may also be placed outside the casing, as a separate piece of equipment.

It will be appreciated that the field user device 10 is equipped with suited power supply, such as a rechargeable battery, to be carried around a field and be powered when operating. In some embodiments, the casing 12 includes a handle and is generally sized and shaped to be carried around a field by a single user. While the display device of the computing device 14 is preferred on the top portion 20 of the casing 12, the other hardware components can be placed elsewhere in the casing 12, or, in some cases, outside of the casing 12.

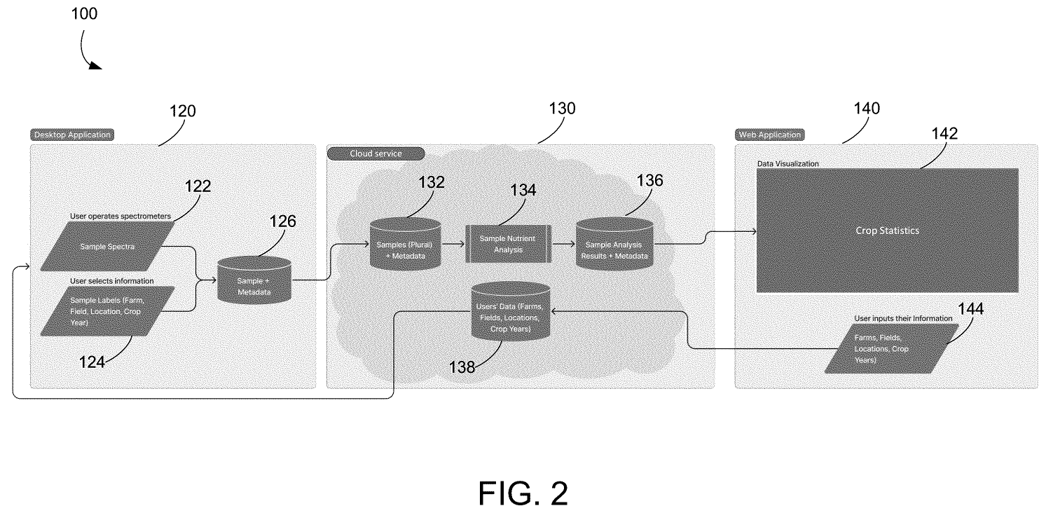

Now referring to FIG. 2, there is shown a system 100 for acquiring a spectrum of a crop sample suited to be implanted with the field user device 10 of FIG. 1. The system 100 includes a desktop application 120, a remote server 130 and a web application 140, all of which are communicatively coupled via a suited means for wireless communication, such as via the internet. The desktop application 120 is configured to interact with the user via a graphical user interface and to receive measurements of the crop sample. The remote server 130 is configured for processing data received from the desktop application 120 and to act as an intermediary between the desktop application 120 and the web application 140. The web application 140 is configured for displaying the results of the analysis and giving a broad and detailed overview of the growth of the crop to the user. In terms of user access, the desktop application 120 along with the web application 140 constitute the frontend part of the system 100 and the remote server 130 constitutes the backend part of the system 100. In some alternative embodiments, the desktop application 120, the remoter server 130 and the web application 140 are all processed on the same computing device. In such cases, the computing device is preferred to be in communication with a database in order to share crop data therewith. It will be appreciated that the analysis on the growth of the crop generally yield the phenotype, genotype, disease, chemistry, and other growth-related information of the like.

The desktop application 120 is configured for prompting the user to obtain various information therefrom. Fundamentally, the desktop application 120 is configured to receive spectra of the crop and crop information. The crop information includes the type of crop, and may also include the location, the farm, the field of the farm, and the date. The term “location” generally refers to the coordinates where the crop sample has been harvested, which can be obtained using the Global Positioning System (GPS). In some cases, the location may also correspond to the location of the field user device. While various types of information may be specified for a given crop, it will be appreciated that the type of crop is usually the information best suited for identifying the crop sample amongst scans of other samples. The spectra are received via a sample spectra module 122, which is usually in communication with one or more spectrometers. The type of spectrometer may vary according to the embodiment, but is configured to yield spectral information relevant to the growth conditions of the crop. The desktop application 120 is also configured to prompt the user to input crop information via a sample label module 124. As such, the sample label module 124 receives the crop information from the user, which enables the spectra obtained by the sample spectra module 122 to be paired with crop information, such as in the form of metadata for instance, which can be processed by a first data combiner 126. It will be appreciated that the term “prompting” referred to herein is representative of a computing device requesting a user to perform an action via the user interface. This may be performed by displaying a text box including text that requests the user to perform the action or by displaying an empty input box that the user may need to fill out before proceeding in the measuring sequence.

The first data combiner 126 is a tool configured to combine into one file the spectrum and the crop information, thereby generating a contextual spectrum of the crop. Indeed, the various crop information provided by the user is used to add data to the sample scanned by the spectrometer. The backend software may subsequently use the crop information to build a model of the crop using the spectral data from the image and the crop information. It will be appreciated that the term contextual refers to the addition of the crop information, which may be representative of the circumstances surrounding the state of the crop when scanning the sample, in the spectrum. In some embodiments, the crop information may be embedded into the file containing the spectrum, such that the crop information is identifiable when processing the spectrum. The embedding may be performed using suited metadata embedding techniques. It will be appreciated that other types of combining techniques may be contemplated, such as concatenating the crop information with the spectrum in a single file, creating a file that contains both the crop information and the spectrum, and the like.

The data produced by the desktop application 120 is provided to the remote server 130, which is configured to process the contextual spectrum and produce an analysis of the spectral content. To do so, the remote server 130 receives the contextual spectrum from the desktop application 120 at a receiving module 132. The receiving module 132 may be configured to pre-process the contextual spectrum and determine if the quality thereof is sufficient to yield a spectral analysis that is representative of the state of the crop. The determination of the quality may vary, and may be based on a predetermined model or be based on a comparison between various spectra. The receiving module 132 may be configured to request the sample spectra module 122 of the desktop application 120 to re-prompt the user to scan the sample for obtaining new spectra, should the spectra received by the receiving module 132 be of insufficient quality. In some embodiments, the receiving module 132 may be configured to request the sample spectra module 122 to re-prompt the user to perform additional scans on the sample for a determined number of iterations to improve the quality of the received contextual spectrum. In some embodiments, the best spectrum from the plurality of spectra is used to form the spectrum. In other embodiments, the multiple spectra may be combined into a single spectrum. The number of spectra may vary, and may be of about 10 to 50 iterations, depending on the quality of the spectra, for instance.

The contextual spectra received by the receiving module 132 are processed by a sample analysis module 134, which is configured to extract various nutrient information, disease information or other growth information from the spectral data. In some embodiments, the sample analysis module 134 may extract the spectral data from the contextual spectrum to improve the analysis efficiency. The person skilled in the art is aware of various techniques to evaluate the level of nutrients (or other growth-related information) found in a spectrum. For instance, the concentration of nitrogen and phosphorus (and other pertinent elements), which can be found by analyzing the spectrum, can correlate to the amount of nutrient found in the soil in which the crop grows, and can indicate the status of growth of the crop. It will be understood that the current technology is not bound to any particular method of spectral analysis, and that any suited method may be implanted to extract crop information from the spectra. The data outputted by the sample analysis module 134 is the analysis results of the spectrum, such as determined nutrient content of the crop and the growth stage thereof.

In one embodiment, a trained artificial intelligence (AI) engine can be used to analyze contextual spectra. More specifically, a plurality of spectra may be acquired from crops which are then also analyzed in a laboratory. The results of the laboratory analysis can be paired to the spectra and fed to the artificial intelligence engine to train it. The higher the quality of the measurements and images, and the greater the number, the greater the reliability of the results which will then be produced by supplying spectra without pre-existing measurements to the engine will be.

In some embodiments in which the spectral data has been removed from the contextual spectrum by the sample analysis module 134, a second data combiner 136 may be configured for combining the crop information with the analysis results to maintain traceability of the state of the crop during the scan. The data outputted by the second data combined 136 is the analysis results combined with crop information for a given contextual spectrum.

In some embodiments, the remote server 130 may include a user data module 138, which is configured for receiving a predetermined crop information selection from the web application 140. The predetermined crop information selection may be representative of a crop model. Consequently, the user data module 138 may be configured to force the sample label module 124 to prompt the user to select predetermined crop information that fits the model stored in the web application.

The web application 140 is configured for receiving and storing the analysis results and the crop information outputted by the second data combiner 136 of the remote server 130. While the remote server 130 is generally used to extract nutrient information from the spectra, the web application 140 is configured for fitting growth model of the crop on the received data. In some embodiments, the remote server 130 may be a cloud service of various kind, such as AMAZON WEB SERVICES™ and the like. For instance, based on given crop information such as the type of crop, the location and the date, and based on a corresponding spectral analysis, the web application 140 is configured for building a model of the crop and making timelines of the growth of the crop, which can be displayed via a data visualization indicator 142. General crop information, such as the type of crop, the locations and the periods at which the crop has been analyzed may be visualized via a crop information module 144.

It will be appreciated that the processing steps performed by the remote server 130 consist of backend operations, i.e. that these operations are generally not meant to be involved by the user operating the field user device. Via the web application, a user, such as a farmer, may access, via e.g. their laptop, desktop, tablet or smartphone computer, for instance, the results of the analysis, and potentially even see the evolution of the results over time, from samples collected earlier in the season, leading to the most recent results. It will be appreciated that while farmers may be a suited type of user for implementing the technology, other types of users may apply.

Turning back to the desktop application 120, it will be understood that this desktop application can include graphical user interface elements displayed on the display device 14 of the field user device 10 shown in FIG. 1.

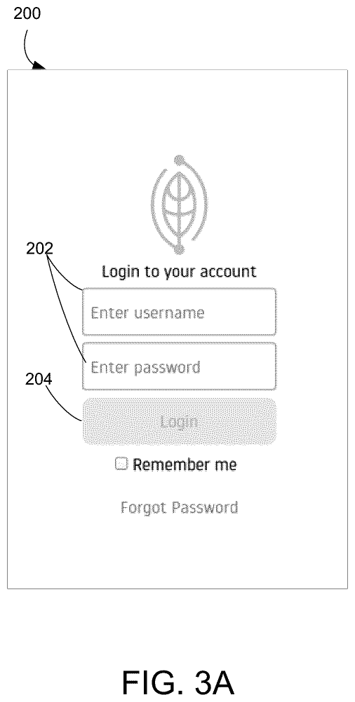

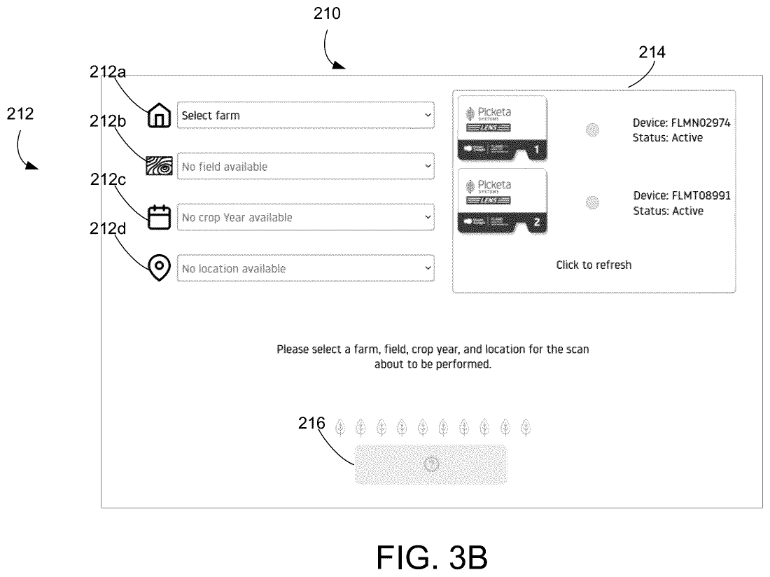

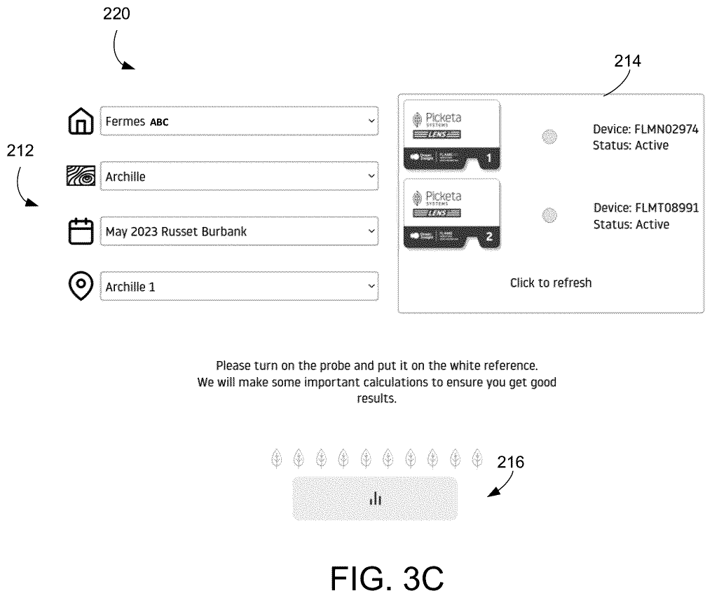

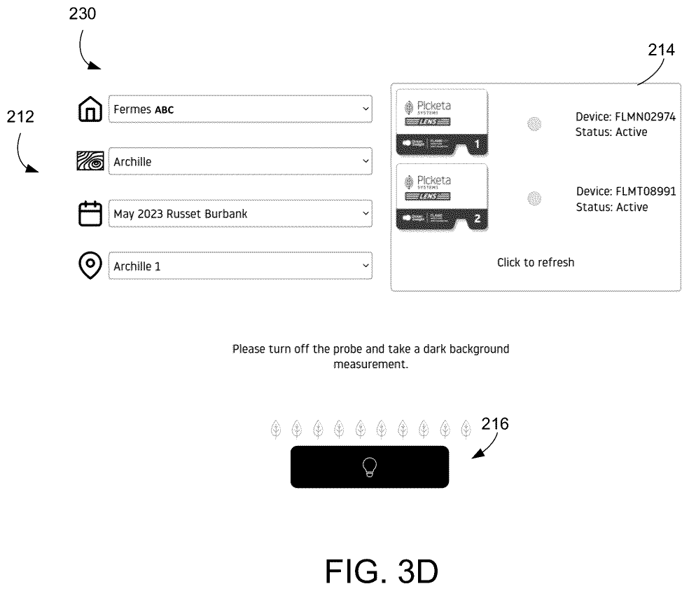

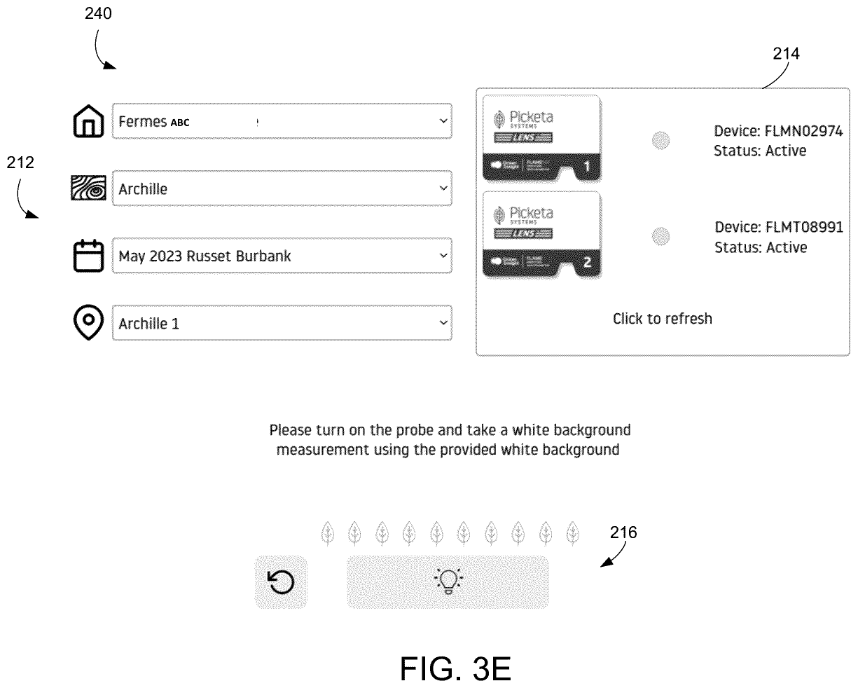

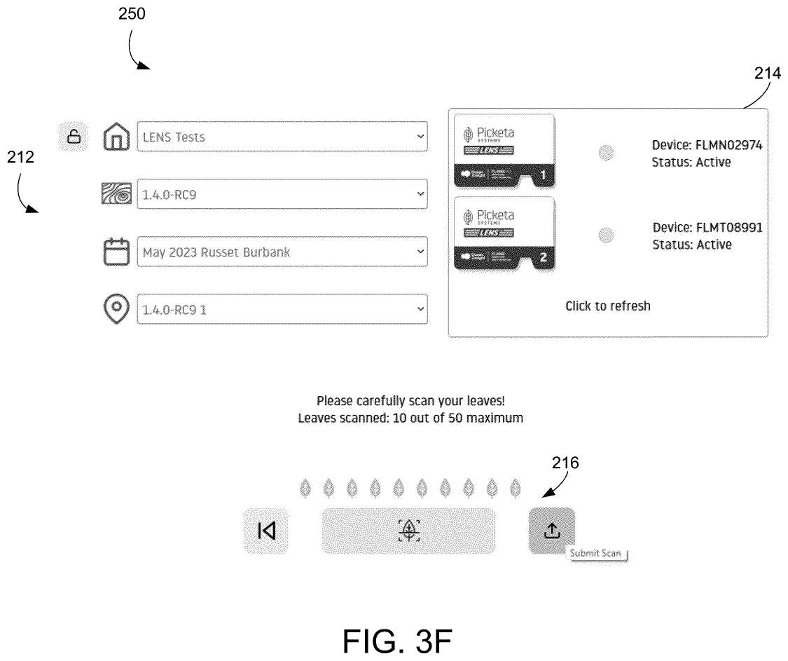

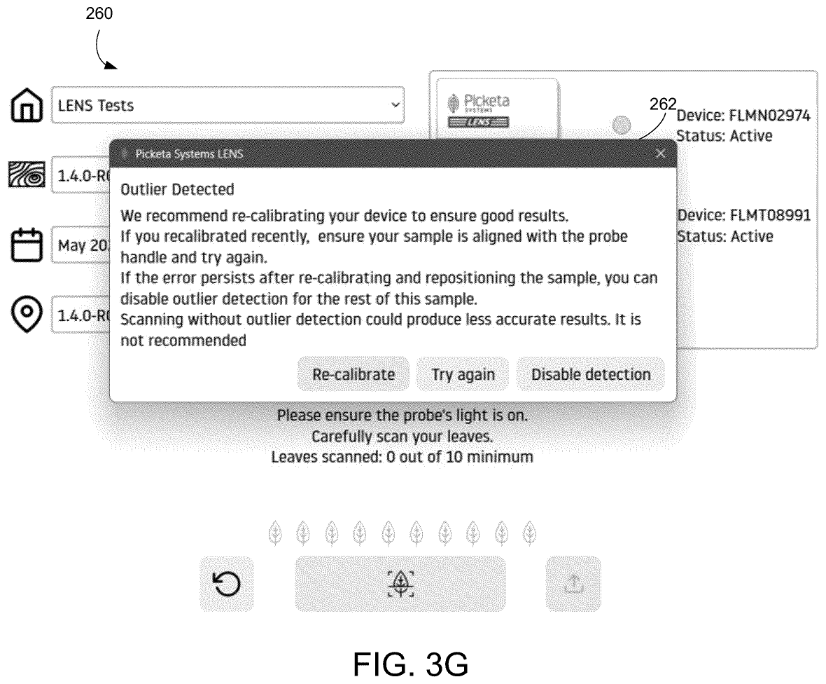

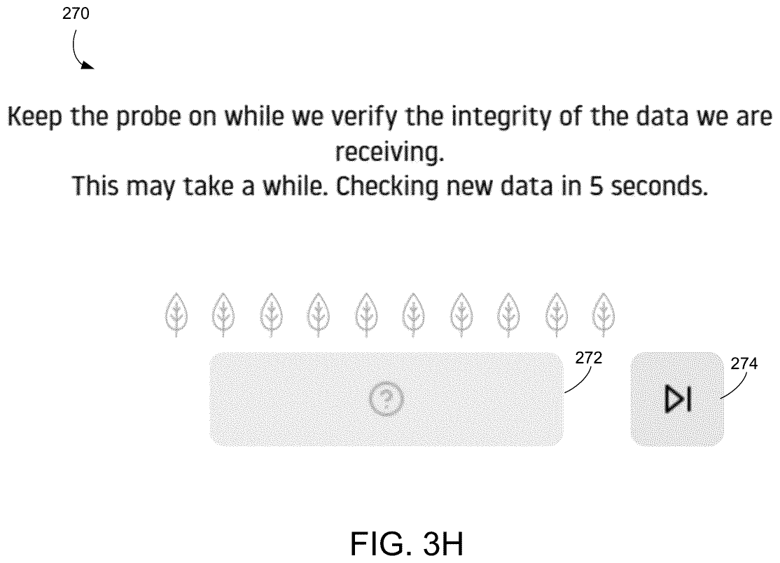

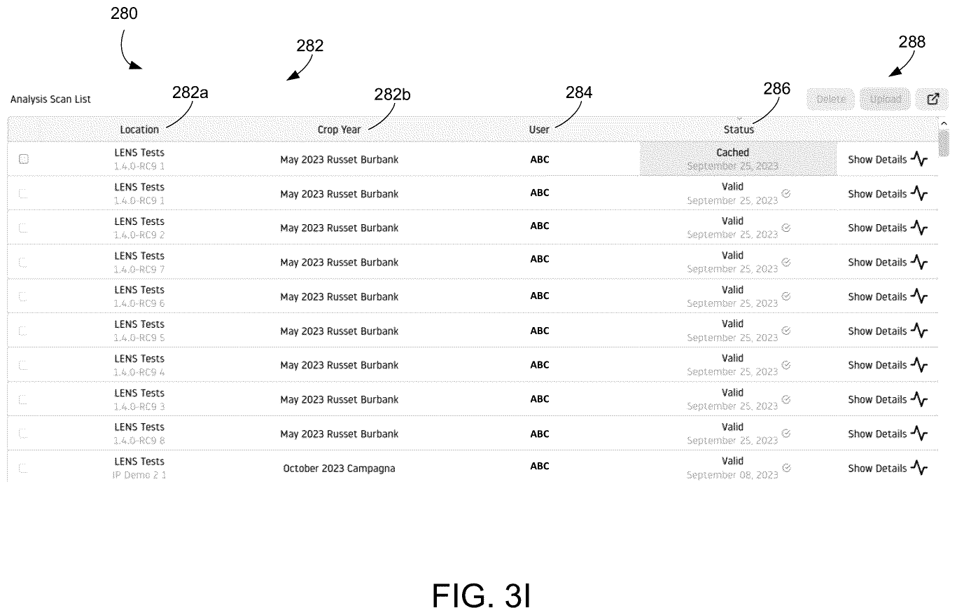

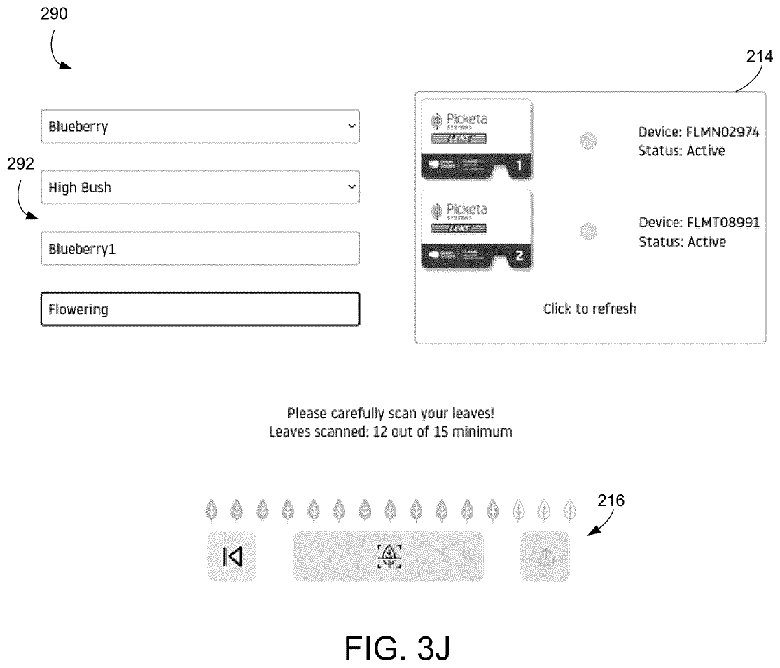

FIGS. 3A-3J place greater emphasis on the elements of the graphical user interface displayed on the display device 14. The sequence of windows presented in FIGS. 3A-3J may therefore be shown on the graphical user interface (GUI) displayed on the display device of the field user device. The first window usually shown to the user is the login window 200 shown in FIG. 3A. The login window 200 presented to the user includes a user credentials element 202, which is configured to prompt the user for credentials such as a username and a password related to the user. This authentication step may be optional, but may securely allow the acquisition system to communicate with the remote server over a telecommunications network such as the Internet without further authentication, for instance. In some other embodiments, the field user device may be used without a wireless communication with a remote server, preventing authentication of the user by matching credentials with the remote server. In such cases, the field user device may be used in an “offline mode”, which consists of a mode where the user inputs credentials that must match credentials stored in the computing device, e.g. credentials that previously matched credentials of the remote server. The field user device may also be used in a “guest mode”, in which the user does not login before scanning the sample. In the “offline mode” and/or the “guest mode”, the user may be required to authenticate with the remote server before uploading the scans.

In some cases, user-related data that includes associated crop data, such as the name of the farm, the previous spectral analysis results in time and the like may be stored in a local or remote database. Access to such user-related data may be contingent upon the user inputting the credentials in the user credential element 202. Once the credentials are received by the computing device, the user may activate login element 204 in the GUI. Upon activation of the login element 204, in the case where the credentials inputted by the user in the user credentials element 202 matches corresponding credentials stored in the database, the session opens for the user.

FIG. 3B shows a crop information page 210 once the session is open, following the login window 200. The information page includes crop information elements 212, a spectrometer status box 214 and a navigation element 216. The crop information elements 212 may comprise, but are not limited to, a farm element 212a, which corresponds to the farm in which the crop is planted, a field element 212b, which corresponds to the field in which the crop is planted, a crop type element 212c, which corresponds to a type of crop and/or a location element 212d, which corresponds to the place where a crop sample is collected. In the embodiment presented as an example, the crop type element allows to specify crop year in addition to a species of the crop, but in some alternate embodiments, the crop type element may allow to specify only a species of crop, or the crop year may be specifiable in a different element. One or more of these elements may be omitted in some embodiments. In some embodiments, the user-related data associated with the user is received by the crop information elements 212, and the user is limited to select the crop information based on predetermined elements found in the user-related data. For instance, the user may be associated with predetermined farms, fields, crop years and scan locations. In this case, the crop information elements 212 may include drop-down lists from which the user selects the crop information. In other embodiments, the user may input directly the crop information in the crop information elements 212 using a keyboard. In such case, the computing device may be configured to prompt the user to re-enter correct crop information should the crop information inputted by the user does not match information on record. Ensuring that the information entered concerning elements such as farm, field, crop, and location corresponds to a predefined string of characters can be particularly helpful in ensuring the accuracy of the subsequent analysis and consistency of the data over time. Accordingly, it can be beneficial in an embodiment to limit the liberty of the user to select one entry amongst a predefined list as opposed to allowing the user to enter a string of characters. Indeed, users are likely to make typos in strings of characters, and a string of character containing a typo may make the other computers which will be in charge of spectral analysis or visual display to the user incapable of performing their intended function.

The spectrometer status box 214 is a feature that displays the status of the spectrometer connected in the field user device. When the field user device is assembled, the computing device may be configured to detect the presence of spectrometers and obtain the status thereof via suited software. For instance, if the spectrometers are successfully connected, the spectrometer status box 214 may display an “Active” status. Conversely, if the computing device is not able to detect the spectrometer, or if errors are detected in the connection, the spectrometer status box 214 may be configured for displaying an error indicative of a failure to connect with the spectrometers. It will be appreciated that the spectrometer status box 214 may be present in various windows displayed in the GUI, and that the status displayed in the spectrometer status box 214 may change if the connection between the spectrometers and the computing device changes.

Once the crop information elements 212 have been filled by the user, and that the spectrometer status box 214 displays an “Active” status, the user is able to activate the navigation element 216 to advance in the sample analysis.

FIGS. 3C-3E show various calibration windows that can be displayed in the GUI for calibrating the spectrometers. As seen in FIG. 3C, there is shown a first calibration window 220 configured for requesting the user to do the first calibration step, which consists of obtaining an integration time for the spectrometer. In order to do so, the first calibration window prompts the user to scan a reference sample that has a high reflectivity in the spectral bands of interest. When scanning the reference sample, the computing device analyzes the measured spectra and obtains an integration time therefrom. It will be appreciated that the integration time corresponds to the amount of time that the detector is allowed to collect photons for a single scan before saturating. The obtained integration time is thus set to correspond to the maximum time possible to collect spectral information for a “near-perfect” reflector. During the first calibration step, the computing device may be configured to set the integration time at a value that correspond to the time necessary to achieve a light intensity reading on the spectrometer that reaches about 80 to 90% of the spectrometer's saturation value. That is, the computing device may be configured for causing the spectrometer to receive light from the probe until at least a portion of the saturation value is obtained in the signal received from the spectrometer. The time taken by the spectrometer to reach the saturation value (or a portion thereof) is thereafter set as the integration time.

Once the integration time has been obtained, the GUI notifies the user that the first calibration step has been completed, and the user is able to change to the next window using the navigation element 216. In some embodiments, the first calibration is performed by the user interacting with the navigation element 216, and the next window is displayed once the integration time has been obtained. In this calibration sequence, the next window corresponds to the second calibration window 230 shown in FIG. 3D. In the second calibration window, the GUI prompts the user to perform a dark background measurement, which will be used as a dark baseline for the measurements. The dark background measurement may be generally performed by turning off the lamp of the spectrometer and measuring the residual signals. In some embodiments, the dark background measurement may be performed by measuring the reference sample, with or without the use of a lamp. Once the dark baseline has been obtained, the GUI notifies the user that the second calibration step has been completed, and the user is able to change to the next window using the navigation element 216. The last window in the calibration sequence is the third calibration window 240 shown in FIG. 3E. In this calibration step, the GUI prompts the user to turn on the lamp of the spectrometers and to take a “white background” measurement using the probe. This measurement may be generally performed by measuring the reference sample and using the obtained integration time and the dark baseline, thereby yielding a white baseline for the spectrometers when the lamp is turned on. Once the third calibration step has been completed, the GUI may prompt the user to move forward to the sample measurements via the navigation element 216. In some embodiment, the navigation element 216 in the second or third calibration window 230, 240 comprises a reset button, in which case the window changes to the previous calibration step, and the GUI displays the first or second calibration window 220, 230, accordingly. Such circumstances may occur when the calibration sequence failed to provide with an adequate dark baseline and/or white baseline.

It will be appreciated that during the calibration sequence, the user may be able to change the crop information in the crop information elements 212. In some embodiments, the calibration sequence may be bypassed, should the calibration of the spectrometers be already performed. In some cases, the calibration parameters may be predetermined using parameters stored in the computing device.

Once the calibration sequence has been processed, the GUI displays the scan window 250, as shown in FIG. 3F. At this step, the GUI prompts the user to scan the sample and to submit the scan via a submit button in the navigation element 216. In some embodiments, the user may be unable to tamper with the crop information elements 212 to avoid unnecessary errors. A lock button may be provided in the crop information elements 212 panel to enable or disable modifications on the crop information elements 212. In some embodiments, the GUI may prompt the user for scanning the sample for a plurality of spectra, e.g., 50 spectra, in order to obtain an average spectrum of the sample, or by performing other statistical aggregation techniques of the like. In some other embodiments, a spectrum may be selected amongst the plurality of spectra based on a predefined model that defines desired spectral properties. In some cases, the plurality of scans may be performed on one or more position on the sample and/or on multiple samples of the same crop for a given location. For instance, the desired spectral properties may be a good spectral resolution across the spectral bands of interest, or a good spectral resolution at wavelengths that are associated with the detection of nutrients and growth conditions. The navigation element 216 in the scan window 250 may include a calibration reset button to reperform the calibration, a delete button configured for deleting a previously acquired scan, a scan button that indicates the computing device that the probe of the spectrometer is in position to scan, thereby causing the spectrometer to acquire a spectrum, and a submit button that causes the computing device to terminate the scan sequence.

In some embodiments, the GUI may present an outlier window 260, as shown in FIG. 3G, in which an outlier box 262 request the user to reperform the calibration of the spectrometer. The outlier box 262 comprises a “Re-calibrate” button, which causes the calibration process to be reperformed when clicked upon, a “Try again” button, which causes the computing device to delete the previously acquired spectrum when clicked upon, and a “Disable detection”, which bypasses the evaluation of the outlier conditions and keeps the previously acquired spectrum, in some cases for the remaining of the scanning sequence, when clicked upon. The outlier box 262 may be displayed should the outlier conditions be met, e.g., the acquired spectrum comprises an error that is detected by an outlier detection algorithm. In some cases, the outlier detection algorithm bases the detection conditions based on previously acquired spectra that are considered acceptable. In some embodiments, the other type of warnings may be provided to the user, such as when the values of the spectra diverge from the baselines set in the calibration sequence, when a predetermined amount of time has passed since the last calibration sequence, after a defined number of spectra have been acquired and the like. The outlier box 262 is generally displayed to the user to avoid measurement errors due to changes in the spectrometers' conditions, or to user-induced errors. The outlier conditions may also serve as recalibration conditions that cause the computing device to prompt the user to recalibrate the spectrometer once those conditions are met.

In some embodiments, the GUI may present a warm-up window 270, as shown in FIG. 3H. The warm-up window 270 is shown prior to the calibration steps, when the lamp is warming up. In this period, the intensity of the light generated by the lamp across the spectral bands of interest generally varies. As such, the GUI requests the user to wait for the lamp to be in a relative steady state once the lamp has warmed up. The warm-up window 270 includes a proceed button 272 that proceeds to the calibration sequence when clicked upon. The proceed button 272 is not operable by the user until the computing device has determined that the lamp parameters have stabilized. The parameters of the lamp that may be the intensity of the light across the spectral bands of interest, the intensity of the light for given wavelengths, the temperature of the lamp and so on. The warm-up window 270 may also include a skip button 274, which enables the user to skip the warm-up step without having stabilized the lamp conditions. In some embodiments, the warm-up time may be of about 15 minutes, depending on the configuration of the field user device.

Once the scans have been obtained by the computing device, the GUI may be configured to display the scans list window 280, as shown in FIG. 31, in which an overview of the scans made by the field user device can be accessed. Each scan in the scan list may correspond to a single scan, or to a value representative of the scan, such as a combination of a plurality of scans. The scans list window 280 has a crop information section 282 having a location column 282a and a crop year column 282b, a user column 284, a status column 286 and an action element 288. In some cases where the computing device is equipped with a means for geolocation (such as a GPS) and an internal clock module, the location column and the crop year column 282b may be automatically filled when the user caches or uploads the scans. For each scan, the crop information section 282 shows crop information relating to the scanned sample, the user column 284 indicates which user has made the scans and the status column 286 indicates whether the scans have been uploaded to the remote server (corresponding to a “Valid” indication in the status column 286) or whether the scans are still stored in the computing device (corresponding to a “Cached” indication in the status column 286). The action element 288 includes a delete button that is configured to delete a scan when clicked upon, an upload button that is configured to upload cached scans and an export button for exporting the scans to another device.

In some embodiments, the GUI may be configured for displaying a research mode window 290, as shown in FIG. 3J, which is an alternative mode than the mode presented in the scan window 250 of FIG. 3F. In the research mode, the GUI prompts the user to input research data in the research data elements 292, which are characteristic to the scanned sampled rather than to the entire crop. The research data may include the type of crop, the subtype of crop, a sample ID and particular features of the crop. Similarly to the scan sequence of scan window 250, the research mode window may be configured to prompt the user to perform a plurality of scans to ensure the liability of the spectra. In the research mode window 290, the user may cause the calibration to be reprocessed, totally or in part, by clicking on the recalibration button of the navigation element 216 and cause the spectrometers to acquire a spectrum using the scan button in the navigation element 216. Once enough scans have been made on the sample, the user may click on the navigation element 216 to submit the scan results.

It will be appreciated that, as opposed to the scan sequence of scan window 250, the results outputted by the research mode window 290 may not be used to contribute to the analysis of the crop growth, but rather to gather growth data for models to be subsequently modelled.

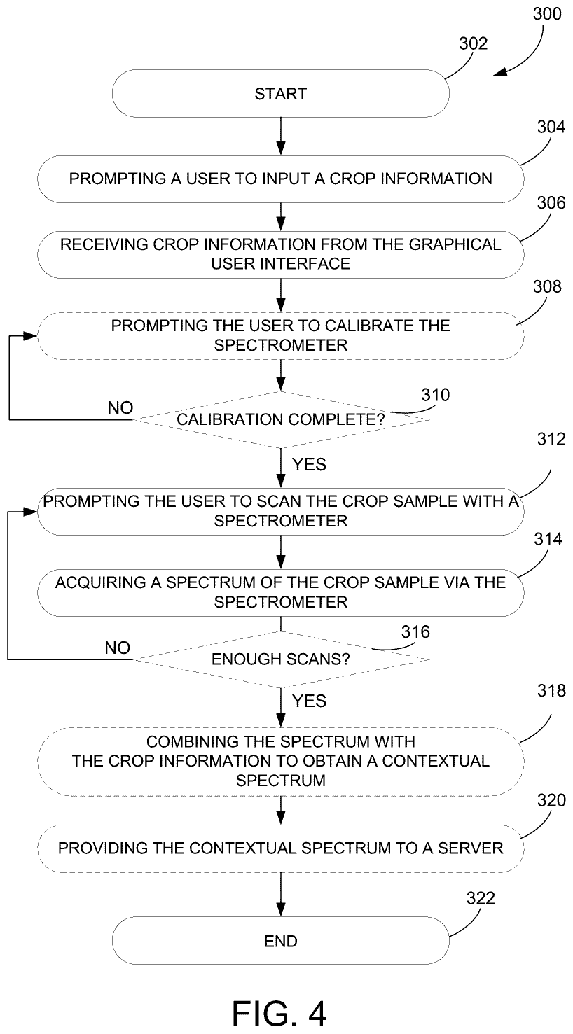

Now referring to FIG. 4, there is shown a flowchart of a method 300 for acquiring a spectrum of a crop sample using the field user device 10 of FIG. 1. The method starts at step 302. At step 304, the user is prompted to input a crop information via a graphical user interface (GUI). During this step, the user provides crop information that is required for monitoring the crop growth using suited models. Further details on the crop information are provided hereabove. At step 306, the crop information is received by the GUI. Once steps 304 and 306 are performed, the scanning steps can be performed.

In some embodiments, the user may be prompted to calibrate the spectrometer prior to said prompting the user to scan the crop sample at step 308. As such, the user may be prompted to perform the calibration steps, such as the calibration steps described above. Once the calibration of the spectrometers has been processed, the calibration may be verified at step 310. Should the calibration fail to be performed adequately, step 310 returns to step 308. Otherwise, the method continues at step 312.

At step 312, the user is prompted to scan the crop sample with the spectrometer, and so by using the probe, for instance. The spectrometer captures the spectra of the crop during this scanning process. Subsequently, at step 314, the computing device acquires the spectrum of the crop sample via the spectrometer.

In some embodiments, once the spectrum has been acquired at step 314, the quality and/or the quantity of the acquired spectrum/spectra may be verified at step 316. Indeed, at step 316, a suited software may be processed on the received spectrum to assess if the quality of the scan is sufficient to be integrated in the analysis of the growth of the crop. Furthermore, if a quantity of spectra is required for forming a spectrum, the quantity of acquired spectra is evaluated at step 316. Should the quality and/or quantity of the acquired spectrum/spectra be insufficient, step 316 returns to step 312. Otherwise, the method may end at step 322 or continue at step 318.

In some embodiments, the spectrum is combined with the crop information to obtain a contextual spectrum, at step 318. It will be appreciated that the crop information embedded as metadata in the spectrum altogether form the contextual spectrum, which can be used to populate the model of the growth of the crop. At step 320, the contextual spectrum is provided to a server, such as a remote server. The method 300 ends at step 322.

In some embodiments, the data used as a selection for the user to select the predetermined crop information is data provided by the company selling the field user device 10, the system 100 at least in part and/or a software having instructions to perform the method 300.

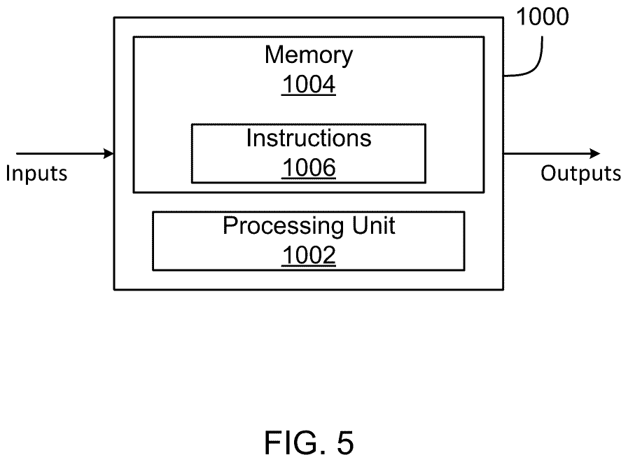

Referring now to FIG. 5, the system 100 of FIG. 2 and/or the method 300 of FIG. 4, may be implemented using a computing device 1000. For simplicity only one computing device 1000 is shown but the system 100 and/or the method 300 may involve more computing devices 1000 which may be the same or different types of devices. The computing device 1000 comprises a processing unit 1002 and a memory 1004 which has stored therein computer-executable instructions 1006. The processing unit 1002 may comprise any suitable devices configured to implement the system 100 and/or the method 300 such that instructions 1006, when executed by the computing device 1000 or other programmable apparatus, may cause the functions/acts/steps of the system 100 and/or the method 300 described herein to be executed. The processing unit 1002 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 1004 may comprise any suitable known or other machine-readable storage medium. The memory 1004 may comprise non-transitory computer readable storage medium, for example, but not limited to, an electronic, magnetic, optical, electromagnetic, infrared, or semiconductor system, apparatus, or device, or any suitable combination of the foregoing. The memory 1014 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 1004 may comprise any storage means (e.g., devices) suitable for retrievably storing machine-readable instructions 1006 executable by processing unit 1002.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure.

Various aspects of the systems and methods described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be apparent to those skilled in the art that changes and modifications may be made without departing from this invention in its broader aspects. The scope of the following claims should not be limited by the embodiments set forth in the examples, but should be given the broadest reasonable interpretation consistent with the description as a whole.

What is claimed is:

1. A method for acquiring a spectrum of a crop sample using an acquisition system having a computer, a spectrometer and a user interface including a display device, the method comprising:
via the display device, prompting a user to input a crop information;
receiving the crop information from the user interface, the crop information including a type of crop;
via the user interface, prompting the user to scan the crop sample with the spectrometer; and
acquiring a spectrum of the crop sample via the spectrometer.

2. The method of claim 1, wherein the display device further includes a graphical user interface, and at least one of said prompting the user to input a crop information and said prompting the user to scan the crop sample is performed via the graphical user interface.

3. The method of claim 1, wherein said prompting a user to input a crop information further comprises prompting the user to select a location amongst a plurality of predetermined locations.

4. The method of claim 1, wherein said prompting the user to input the crop information further comprises prompting the user to select the type of crop amongst a plurality of predetermined types of crop.

5. The method of claim 1, further comprising prompting the user to calibrate the spectrometer prior to said prompting the user to scan the crop sample, the calibration comprising obtaining at least one of an integration time, a dark baseline and a white baseline for the spectrometer.

6. The method of any one of claim 1, further comprising combining the spectrum with the crop information to obtain a contextual spectrum.

7. The method of claim 1, wherein the spectrometer is configured to operate in a spectral band defined between about 350 nm and 2,500 nm.

8. The method of claim 1, wherein the prompting the user to scan the crop sample comprises prompting the user to scan the crop sample more than once, thereby obtaining a plurality of spectra of the crop sample.

9. The method of claim 1, further comprising activating a lamp prior to said prompting the user to scan the crop sample, and prompting the user to wait for a predetermined amount of time until parameters of the lamp stabilize.

10. The method of claim 1, further comprising prompting the user to recalibrate the spectrometer once a recalibration condition has been obtained.

11. The method of claim 10, wherein the recalibration condition comprises at least one of a predetermined amount of time that passed after a previous calibration, a predetermined number of spectra acquired after the previous calibration and a detection of an outlier condition.

12. The method of claim 6, wherein said combining the spectrum with the crop information is performed by embedding metadata corresponding to the crop information in a file that includes the spectrum.

13. The method of claim 8, wherein said acquiring the spectrum comprises combining the plurality of spectra of the crop sample.

14. The method of claim 8, wherein said acquiring the spectrum comprises selecting a desired spectrum amongst the plurality of spectra of the crop sample, the desired spectrum having properties that correspond at least partially to a desired spectrum.

15. The method of claim 6, further comprising providing the contextual spectrum to a server, the server being configured for analyzing the contextual spectrum and obtaining conditions of the crop therefrom.

16. The method of claim 15, wherein the conditions of the crop comprises at least one of a type and concentration of at least one type of nutrient, a presence of disease and a stage of growth.

17. The method of claim 15, further comprising receiving predetermined crop information from the server, wherein said prompting a user to input a crop information is based on the predetermined crop information.

18. The method of claim 15, further comprising storing the spectrum on a storage device prior to said providing the spectrum to the server.

19. The method of claim 1, wherein the crop information comprises research data for the crop sample.

20. The method of claim 1, wherein the spectrometer includes two or more spectrometers each covering at least partially a respective spectral band.
